(19) United States
(12) Patent Application Publication
(10) Pub. No.: US 2014/0276110 A1
(43) Pub. Date: Sep. 18, 2014

(54) IMAGING GUIDEWIRE SYSTEM WITH FLOW VISUALIZATION

(71) Applicant: VOLCANO CORPORATION, San Diego, CA (US)

(72) Inventor: Paul Hoseit, El Dorado Hills, CA (US)

(73) Assignee: VOLCANO CORPORATION, San Diego, CA (US)

(21) Appl. No.: 14/202,936

(22) Filed: Mar. 10, 2014

Related U.S. Application Data
(60) Provisional application No. 61/783,684, filed on Mar. 14, 2013.

(51) Int. Cl. A61B 5/00; A61B 5/0215; A61B 5/026

(52) U.S. Cl. CPC A61B 5/6851; A61B 5/0261; A61B 5/02154
USPC 600/478

(57) ABSTRACT

The invention is a system comprising a guidewire having expanded imaging capabilities and a processor for processing the image data and causing relevant information, such as flow, to be displayed. The system is configured to cause image data to be processed and reconfigured in a user friendly format, e.g., color-coded, to provide details of flow and device placement within a biological lumen.

### RELATED APPLICATION

This application claims priority to U.S. Provisional Application No. 61/783,684, filed Mar. 14, 2013, which is incorporated by reference in its entirety.

### FIELD OF THE INVENTION

The invention relates to systems for evaluating cardiovascular health by imaging a patient's vasculature. In particular, the invention relates to systems including an imaging guidewire being capable of measuring fluidic flow and/or device placement.

### BACKGROUND

Access guidewires are used in the vasculature or other anatomical passageways to guide other devices, e.g., a catheter, to a location. Typically, the guidewire is inserted into an artery or vein and guided through the vasculature under fluoroscopy (real time x-ray imaging) to the location of interest. Then one or more devices are delivered over the guide wire to diagnose, image, or treat tissues at the location.

Crossing guidewires are used in the vasculature or other anatomical passageway to pass through, and/or around, blockages or narrowed passages in the anatomical passageway, hence the name “crossing.” Crossing guidewires are typically stiffer than access guidewires to provide better tracking and the ability to deliver lateral force at the distal end by pushing on the proximal end. Like access guidewires, crossing guidewires are also guided using fluoroscopy. Both access and crossing guidewires can be collectively referred to as “guidewires.”

Advances in materials and miniaturization have made it possible to include sensors on guidewires, such as pressure and flow sensors. For example, the FLOWIRE® Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. These improvements have greatly improved patient care because it is now possible to obtain relevant clinical information during guidewire placement, or during a crossing procedure.

While available guidewires can achieve some flow measurements, it would be preferable to have a guidewire with full imaging capabilities, allowing a physician to evaluate vasculature and implants that may have been placed therein. Currently, evaluating the placement of a stent, for example, requires the extra step of placing an imaging catheter for evaluating the stent after the stent has been deployed with a separate catheter. Each catheter exchange, however, increases the length of a surgical procedure while subjecting the patient to additional risks, such as arterial or venous perforation or dislodgement of thrombus as the multiple catheters are inserted and removed.

### SUMMARY

The invention is a system comprising a guidewire having expanded imaging capabilities and a processor for processing the image data and causing relevant information, such as flow, to be displayed. The system is configured to cause image data to be processed and reconfigured in a user friendly format, e.g., color-coded, to provide details of flow and device placement within a biological lumen.

An additional benefit of the invention is that it allows flow and structure evaluation in lumens that are too small for imaging catheters, i.e., the instrument more typically used to image vasculature. Ideally, a guidewire of the system is small, on the order of 1 mm or smaller, allowing the guidewire to be placed throughout the vasculature, as well as the lymphatic, urological, and reproductive systems. Because of this versatility, the system can be used to treat a number of organs, such as the kidneys, lungs, brain, heart, pancreas, ovaries, or testes. Combined flow and structure images can be particularly useful in evaluating previously-placed interventional structures, such as stents.

Additionally, because therapeutic catheters may be used in conjunction with guidewires of the system, the guidewire can be left in place during the procedure. This allows imaging and characterizing of the interventional area before and after therapy or other procedure, e.g., thrombus removal. Accordingly, procedure times are shortened, resulting in a reduction of the amount anesthesia, contrast, and x-rays to which a patient is exposed. For example, in an endovascular procedure, the guidewire can be placed once using angiography, the treatment site imaged and evaluated using the system, a therapy administered, and the treatment site subsequently re-imaged and evaluated with the system to confirm the results of the treatment.

The invention achieves its versatility by employing a guidewire comprising optical fibers bundled to a core. The design makes efficient use of optical Bragg gratings that work as partially- or fully-reflective wavelength-selective elements. One portion of the fibers is coupled to one or more photoacoustic transducers that convert electromagnetic radiation into acoustic energy, and one portion of the fibers is coupled to one or more acoustic-sensing materials, for example photoreflective material or materials arrange in a strain-gauge-type configuration. The invention additionally uses image-processing algorithms to identify structures, such as lumen borders and stent arms, and present them in an easy-to-understand format.

In an embodiment, the system comprises a guidewire including a first optical fiber having a first blazed Bragg grating, a photoabsorptive member, and a sensor. The first blazed Bragg grating is designed to be at least partially reflective of a first wavelength. The photoabsorptive member absorbs the first wavelength and is in photocommunication with the first blazed Bragg grating. The invention additionally lends itself to methods of treating a subject, including imaging a subject with acoustic energy produced from a guidewire, and optionally measuring a fluidic pressure with a pressure sensor coupled to the guidewire.

### BRIEF DESCRIPTION OF THE FIGURES

FIG. 1A depicts a distal end of an embodiment of a guidewire having a pressure sensor integrated into the distal tip;

FIG. 1B depicts the simultaneous or sequential delivery and reception of acoustic waves (curved lines) from the distal end of the embodiment of a guidewire of FIG. 1A;

FIG. 2 depicts an embodiment of a system for ultrasound imaging comprising a guidewire, a display, a detector, a controller, and image processing;

FIG. 3 is a block diagram of an exemplary system for identifying flow and/or structure in an image acquired with an imaging guidewire and displaying relevant flow and/or structure information;

FIG. 4 is a block diagram of an exemplary system for identifying flow and/or structure in an image acquired with an imaging guidewire and displaying relevant flow and/or structure information;

FIG. 5 is a block diagram of an exemplary system for identifying flow and/or structure in an image acquired with an imaging guidewire and displaying relevant flow and/or structure information;

FIG. 6 shows a block diagram of an algorithm to identify relevant flow and/or structural information in an image acquired with an imaging guidewire of the system;

FIG. 7 depicts a false-color image of a blood vessel indicating healthy blood flow;

FIG. 8 depicts a false-color image of a blood vessel with a malapposed stent.

### DETAILED DESCRIPTION

The present invention is a system comprising a guidewire capable of imaging and flow measurement and a processor configured to process the image data from the guidewire. The guidewire comprises optical fibers and photoabsorptive and/or photoreflective materials and the system is capable of making intravascular ultrasound (IVUS) measurements to determine flow and structures, e.g., a stent, in proximity to the guidewire. The disclosed invention will improve interventional evaluation by providing a physician with critical information about flow and structure while also reducing the time for procedures. In a further embodiment, the system is useful for evaluating intravascular structures to determine an appropriate location for the placement of an implantable structure, e.g., a stent, and/or and the efficacy of prior treatments.

Guidewires typically have diameters of 0.010″ to 0.035″, with 0.014″ being the most common. Guidewires (and other intravascular objects) are also sized in units of French, each French being ⅓ of a mm or 0.013″. Guidewire lengths vary up to 400 cm, depending on the anatomy and work flow of the procedure. The ends of the guidewire are denoted as distal (far from the user, i.e., inside the body) and proximal (near the user, i.e., outside the body). Often a guidewire has a flexible distal tip portion about 3 cm long and a slightly less flexible portion about 30 to 50 cm long leading up to the tip with the remainder of the guidewire being stiffer to assist in maneuvering the guidewire through tortuous vasculature, etc. The tip of a guidewire typically has a stop or a hook to prevent a guided device, e.g., a catheter from passing beyond the distal tip. In some embodiments, the tip is deformable to allow a user to produce a desired shape.

Advanced guidewire designs include sensors that measure flow and pressure, among other things. For example, the FLOWIRE® Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. Additionally, the PRIMEWIRE® pressure guidewire, available from Volcano Corp. (San Diego, Calif.), provides a microfabricated microelectromechanical (MEMS) pressure sensor for measuring pressure environments near the distal tip of the guidewire. Additional details of guidewires having MEMS sensors can be found in U.S. Patent Publication No. 2009/0088650, incorporated herein by reference in its entirety.

The proximal end of a guidewire varies in construction depending upon the complexity of the device. Simple guidewires, used for placement of devices such as catheters, are untethered, i.e., the proximal end does not need to be connected to other equipment. Sensing guidewires, on the other hand, require a signal connection when the sensor is used. The signal connection is typically detachable to facilitate loading/unloading catheters, however it is also possible to load a rapid exchange catheter on a guidewire prior to guidewire insertion. Placement guidewires without tethers are less expensive, and most useful when a procedure requires multiple catheter exchanges, because each catheter can be quickly removed from the guidewire and the next catheter placed on the guidewire.

While not shown in detail in the figures, a sensing guidewire (like the invention) has a tethered proximal end, typically with a detachable connection. As discussed below, guidewires of the invention use optical fibers to supply light to the distal end of the guidewire and to detect returning light. Accordingly, guidewires of the invention have a tether comprising optical fibers and one or more detachable optical couplings. In some embodiments, all of the optical fibers of the guidewire are coupled into a single optical coupling. The tethers may additionally comprise electrical connections, as needed, to produce acoustic energy or to receive acoustic echoes.

Additionally, while not shown in detail in the figures, a guidewire of the invention has a mid-body connecting the proximal and distal ends. The mid-body is typically a length between 50 and 500 cm, typically greater than or equal to 100 cm, typically less than or equal to 400 cm, typically about 200 to 300 cm. The mid-body typically has a core, which is typically a biocompatible and resilient metal wire. Often the core comprises a coil that provides stiffness while avoiding kinking when traversing tortuous vasculature. The core may comprise multiple strands of metal fiber or the core may be a unitary piece of metal wire. The core is typically constructed from Nitinol or stainless steel. As discussed in greater detail below, the mid-body will also comprise a number of optical fibers to deliver light to the distal end of the guidewire and to return reflected light. The optical fibers may be bound to the core with adhesive or fasteners. The optical fibers may be touching the core or the optical fibers may be displaced axially from the core with spacer, typically a resilient polymer. The core and the optical fibers (and optionally spacer) are coated with a coating to help the guidewire pass through an introducer, to pass through the vasculature, and to help delivered devices (e.g., catheter) easily pass over the guidewire. In addition to being both biocompatible and resilient (will not dislodge or flake), the guidewire coating is typically lubricious to reduce the friction between the guidewire and a catheter.

The sensors incorporated into a guidewire of the invention can be of a variety of structures small enough to be incorporated into a guidewire and suitable for pressure sensing in an anatomical environment, e.g., an artery or vein. A guidewire mounted pressure sensor may be, for example, a MEMS sensor manufactured using deep reactive ion etching (DRIE) to form the solid-state sensor rather than previously used mechanical saws. DRIE is a highly anisotropic etch process for creating deep, steep-sided holes and trenches in solid-state device wafers, with aspect ratios of 20:1 or more. DRIE was originally developed for MEMS structures such as cantilever switches and microgears. However, DRIE is also used for producing other devices such as to excavate trenches for high-density capacitors for DRAM. DRIE is capable of fabricating 90° (truly vertical) walls. Using DRIE leads to a number of new pressure sensor designs for intravascular applications wherein the sensor is mounted at a distal end of a pressure measuring coronary guidewire.

A distal end of an embodiment of a guidewire 100 suitable for use in a system of the invention is depicted in FIG. 1A. The guidewire 100 comprises optical fibers 110. Optical fibers 110 may be constructed from glass or plastic. Optical fibers 110 include blazed Bragg gratings 115 (discussed below). In the embodiment shown in FIG. 1A, the blazed Bragg gratings 115 of the optical fiber 110 are in proximity to ultrasound transducers 120. The ultrasound transducers 120 may also comprise a photoreflective element that is deflected with the receipt of incident acoustic waves. In other embodiments, the ultrasound transducer and photoreflective elements are separate structures, however it is to be understood that ultrasound transducer 120 refers to a stand-alone ultrasound transducer, a combined ultrasound transducer and photoreflective element, or a stand-alone photoreflective element. The guidewire 100 terminates in a tip 150. The core of the guidewire is not shown in FIG. 1A to assist clarity, however, a core is typically present in the guidewire 100, as discussed above.

The guidewires of the invention employ fiber Bragg gratings to couple light into or out of the optical fibers 110. A fiber Bragg grating is a periodic modulation of the index of refraction in a fiber. When the periodicity, d, of the modulation satisfies the Bragg condition (d=nλ/2) for a wavelength λ, that wavelength will be reflected. That is, the fiber Bragg grating acts as a wavelength-selective mirror. The degree of index change and the length of the grating influences the ratio of light reflected to that transmitted through the grating. A review of fiber Bragg gratings can be found at A. Othonos, Rev. Sci. Inst., 68 (12), 4309 (1997), incorporated by reference herein in its entirety. The optical fibers 110 comprise a normal Bragg grating (back reflective—not shown in FIG. 1A) in addition to blazed Bragg gratings (angle reflective) 115. Blazed Bragg gratings are discussed in greater detail in Othonos, referenced above.

As shown in FIG. 1B, the blazed Bragg gratings couple light, 160, from the optical fibers 110, out of the fibers and into an ultrasound transducer 120. The light 160 originates in a light source, discussed in detail below. As shown in FIG. 1B, the light 160 coupled out of the first optical fiber 110 by the blazed Bragg grating 115 will impinge on the ultrasound transducer 12( ) producing outbound ultrasonic waves 180. The outbound ultrasonic waves 180 are then absorbed, reflected, and scattered by structures, e.g., tissues, surrounding the ultrasonic transducer 120. The inbound ultrasonic waves 190 reflected from the structures are received by the ultrasonic transducer 120, resulting in a deflection of photoreflective materials (not shown). The change in a pathlength between the photoreflective material and the blazed Bragg grating results in a signal that can be used to image the tissue surrounding the device (discussed in detail below). In some embodiments, a similar structure of blazed Bragg gratings 115 and ultrasonic transducers 120 can be used to make Doppler measurements, e.g., of a flowing fluid, e.g., blood.

In an embodiment, the ultrasound transducer 120 comprises an optically-absorptive photoacoustic material, which produces ultrasound waves 180 when it absorbs light 160. The optically absorptive photoacoustic material is positioned, with respect to the blazed Bragg grating 115, to receive the optical energy leaving the blazed grating. The optically absorptive photoacoustic material is selected to absorb light 160, and produce and transmit ultrasound or other acoustic waves for acoustic imaging of a region of interest about the distal tip of the guidewire 100. The acoustic waves generated by the photoacoustic material interact with tissues vasculature) in the vicinity of the distal end of the guidewire 100, and are reflected back (echoes). The reflected acoustic waves are collected and analyzed to obtain information about the distance from the tissues to the guidewire, or the type of tissue, or other information, such as blood flow or pressure.

As discussed above, the ultrasound transducer 120 may comprise a photoreflective element to receive reflected acoustic waves. The photoreflective member is flexibly resilient, and is displaced by acoustic waves reflected by the tissues. A transparent (or translucent) flexible material is disposed between the optical fiber 110 and the photoreflective material of the ultrasound transducer 120, thereby allowing a deflection in the photoreflective material to change the path length of the light between the optical fiber 110 and the photoreflective material. In alternative embodiments, a void can be left between the optical fiber 110 and the photoreflective material.

In the absence of incident acoustic energy, the photoreflective material will be in a neutral position, providing a baseline path length between the optical fiber 110 and the photoreflective material. Incident light, transmitted via the optical fiber 110, will be reflected from the photoreflective material, and return to a detector at the proximal end of the guidewire (not shown) with a characteristic round trip time. The light transmitted via the optical fiber 110 may be the same light as used to produce acoustic energy (discussed above), the same light used to photoactivate therapeutics (discussed above), or a different light (wavelength, pulse frequency, etc.) may be used. When the photoreflective material is deflected, i.e., with the absorbance of incident acoustic waves, the path length between the third optical fiber 110 and the photoreflective material will change, resulting in a measurable change in the properties of the reflected light, as measured by a detector at the proximal end of guidewire (not shown). The change may be a shift in the time of the return trip, or the shift may be an interferometric measurement. The change in the properties of the reflected light can then be analyzed to determine properties of the tissues from which the acoustic waves were reflected.

In some embodiments, the incident light 160 is pulsed at a frequency at which the acoustic waves will be produced. Light sources that produce pulses at ultrasonic frequencies, e.g., 1 MHz and greater, are commercially-available, typically solid state lasers. Nonetheless, photoacoustic materials have natural acoustic resonances, and the photoacoustic material will naturally produce a spectrum of acoustic frequencies when the material absorbs the incident light 160 and the photoacoustic material relaxes by producing acoustic waves. If it is desired to rely on the natural frequencies of the photoacoustic material, the incident light 160 may be continuous.

In an embodiment, the photoacoustic material has a thickness in the direction of propagation that increases the efficiency of emission of acoustic energy. In some embodiments, the thickness of the photoacoustic material is selected to be about one fourth of the acoustic wavelength of the material at the desired acoustic frequency (“quarter wave matching”). Providing photoacoustic material with quarter wave matching improves the generation of acoustic energy by the photoacoustic material, resulting in improved ultrasound images. Using the quarter wave matching and sensor shaping techniques, the productivity of the fiber blazed Bragg sensor and photoacoustic materials approaches the productivity of piezoelectric transducers known in the field of ultrasound imaging.

In one embodiment, before the photoacoustic transducer is fabricated, the guidewire 100 is assembled, such as by binding the optical fibers 110 to the core (not shown) and tip 150, and optionally coating the guidewire 100. The photoacoustic transducer 120 is then integrated into the guidewire 100 by etching or grinding a groove in the assembled guidewire 100 above the intended location of the blazed Bragg grating 115 in the first optical fiber 110. As discussed above, the depth of the groove in the assembled guidewire 100 can play a role in the efficiency of the acoustic wave production (e.g., quarter wave matching).

Once the photoacoustic transducer 120 location has been defined, the blazed Bragg grating 115 can be added to the first optical fiber 110. In one example, the grating 115 is created using an optical process in which the portion of the first optical fiber 110 is exposed to a carefully controlled pattern of UV radiation that defines the blazed Bragg grating 115. After the blazed Bragg grating is complete, a photoacoustic material is deposited or otherwise added over the blazed Bragg grating 115 to complete the transducer 120. An exemplary photoacoustic material is pigmented polydimethylsiloxane (PDMS), such as a mixture of PDMS, carbon black, and toluene. The photoacoustic materials may naturally absorb the light 160, or the photoacoustic material may be supplemented with dyes, e.g., organic dyes, or nanomaterials (e.g., quantum dots) that absorb light 160 strongly. The photoacoustic material can also be “tuned” to selectively absorb specific wavelengths by selecting suitable components.

In another embodiment, not shown in the figures, the optical fibers 110 may be modified to include first and second normal Bragg gratings. These first and second normal Bragg gratings are partially and mostly reflective, respectively, and create a resonant cavity in the optical fiber 110. In the absence of incident acoustic energy, light in the resonant cavity has a characteristic return signature, e.g., an optical decay signal. With the incidence of reflected acoustic energy, the path length and/or path direction of the resonant cavity will be modified, leading to a change in the return signature. By monitoring changes in the return signature, it is possible to determine the timing of reflected acoustic signals, and hence, properties of the tissues from which the acoustic waves were reflected. The detection is similar to known methods of detecting strain or temperature changes with optical fibers.

In one example of operation of this alternate embodiment, light reflected from the blazed Bragg grating 115 excites the photoacoustic material 120 in such a way that the optical energy is efficiently converted to substantially the same acoustic frequency for which the resonant cavity sensor is designed. The blazed Bragg grating 115 and the photoacoustic material 120, in conjunction with the resonant sensor, provide both an acoustic transducer and a receiver, which are harmonized to create an efficient unified optical-to-acoustic-to-optical transmit/receive device. In some embodiments, more than one type of light (e.g., wavelength) can be coupled into the same fiber, allowing one to be used to produce the acoustic wave and another to monitor reflected acoustic waves. In a further example, the optical transmit/receive frequencies are sufficiently different that the reception is not adversely affected by the transmission, and vice-versa.

Any of the guidewires described above may be part of a system for imaging and identifying flow and structures. An exemplary system 200 is shown in FIG. 2. The system includes a guidewire 100 having an optical fiber 614 coupled to the proximal end, allowing a source of light 620 to be coupled into the optical fiber. Of course, multiple optical fibers may be coupled into a single fiber, such as 614, to facilitate signal production and detection. The source of light 620 may be coupled or split with fiber couplers, dichroics, and filters as necessary to achieve the desired performance. Furthermore, a particular fiber need not be limited to a single light source, as some fibers can support multiple wavelengths simultaneously and the wavelengths can be separated for analysis using known multiplexing techniques.

The source of light 620 for the system 200 may be any known light source capable of producing light with the desired temporal and frequency characteristics. Source 620 may be, for example, a solid-state laser, a gas laser, a dye laser, or a semiconductor laser. Sources 620 may also be an LED or other broadband source, provided that the source is sufficiently powerful to drive the photoacoustic transducers. In some instances the sources 620 is gated to provide the needed temporal resolution. In other instances, the source 620 inherently provides short pulses of light at the desired frequency, e.g., 20 MHz.

A detector 340, coupled to fiber 616 is used to measure changes to the coupled light to determine how the acoustic environment of the guidewire 100 is changing. The detector may be a photodiode, photomultiplier tube, charge coupled array, microchannel detector, or other suitable detector. The detector may directly observe shifts in return light pulses, e.g., due to deflection of the photoreflective material, or the detector may observe interferometric changes in the returned light due to changes in pathlength or shape. Fourier transformation from time to frequency can also be used to improve the resolution of the detection.

As shown in FIG. 2, a controller 650 will be used to synchronize the source 620 and the detector 340. The controller may maintain system synchronization internally, or the system may be synchronized externally, e.g., by a user. The output of the detector 340 will typically be directed to image processing 360 prior to being output to a display 380 for viewing. As discussed below, the image processing will deconvolve the reflected light to produce distance and/or tissue measurements, and those distance and tissue measurements can be used to produce an image, for example an intravascular ultrasound (IVUS) image. The image processing may additionally include spectral analysis, i.e., examining the energy of the returned acoustic signal at various frequencies. Spectral analysis is useful for determining the nature of the tissue and the presence of foreign objects. A plaque deposit, for example, will typically have a different spectral signature than nearby vascular tissue without such plaque, allowing discrimination between healthy and diseased tissue. Also a metal surface, such as a stent, will have a different spectral signal. Such signal processing may additionally include statistical processing (e.g., averaging, filtering, or the like) of the returned ultrasound signal in the time domain. Other signal processing techniques known in the art of tissue characterization may also be applied.

Other image processing may facilitate use of the images or identification of features of interest. For example, the border of a lumen may be highlighted or plaque deposits may be displayed in a visually different manner (e.g., by assigning plaque deposits a discernible color) than other portions of the image. Other image enhancement techniques known in the art of imaging may also be applied. In a further example, similar techniques can be used to discriminate between vulnerable plaque and other plaque, or to enhance the displayed image by providing visual indicators to assist the user in discriminating between vulnerable and other plaque. Other measurements, such as flow rates or pressure may be displayed using color mapping or by displaying numerical values.

A system of the invention may be implemented in a number of formats. An embodiment of a system 300 of the invention is shown in FIG. 3. The core of the system 300 is a computer 360 or other computational arrangement comprising a processor 365 and memory 367. The memory has instructions which when executed cause the processor to determine a baseline measurement prior to conducting a therapeutic procedure and determine a post-therapy measurement after conducting the therapeutic procedure. The instructions may also cause the computer to compare the post-therapy measurement to the baseline measurement, thereby determining the degree of post-therapy improvement after conducting the therapeutic procedure. In the system of the invention, the physiological measurement data of vasculature will originate with a guidewire 100 as discussed above, whose signal is collected with detector 340. Having collected the image data, the processor then processes the data to build images and identify flow and/or structures and then outputs the results. The results are typically output to a display 380 to be viewed by a physician or technician.

In advanced embodiments, system 300 may comprise an imaging engine 370 which has advanced image processing features, such as image tagging, that allow the system 300 to more efficiently process and display intravascular and angiographic images. The imaging engine 370 may automatically highlight or otherwise denote areas of interest in the vasculature. The imaging engine 370 may also produce 3D renderings or other visual representations of the physiological measurements. In some embodiments, the imaging engine 370 may additionally include data acquisition functionalities (DAQ) 375, which allow the imaging engine 370 to receive the physiological measurement data directly from the catheter 325 or collector 347 to be processed into images for display.

Other advanced embodiments use the I/O functionalities 362 of computer 360 to control the detector or to trigger the light source for the guidewire. While not shown here, it is also possible that computer 360 may control a manipulator, e.g., a robotic manipulator, connected to catheter 325 to improve the placement of the guidewire 100.

A system 400 of the invention may also be implemented across a number of independent platforms which communicate via a network 409, as shown in FIG. 4. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

As shown in FIG. 4, the detector 340 facilitates obtaining the data, however the actual implementation of the steps can be performed by multiple processors working in communication via the network 409, for example a local area network, a wireless network, or the internet. The components of system 400 may also be physically separated. For example, terminal 467 and display 380 may not be geographically located with the intravascular detection system 320.

As shown in FIG. 4, imaging engine 859 communicates with host workstation 433 as well as optionally server 413 over network 409. In some embodiments, an operator uses host workstation 433, computer 449, or terminal 467 to control system 400 or to receive images. An image may be displayed using an I/O 454, 437, or 471, which may include a monitor. Any I/O may include a monitor, keyboard, mouse, or touch screen to communicate with any of processor 421, 459, 441, or 475, for example, to cause data to be stored in any tangible, nontransitory memory 463, 445, 479, or 429. Server 413 generally includes an interface module 425 to communicate over network 409 or write data to data file 417. Input from a user is received by a processor in an electronic device such as, for example, host workstation 433, server 413, or computer 449. In certain embodiments, host workstation 433 and imaging engine 855 are included in a bedside console unit to operate system 400.

In some embodiments, the system may render three dimensional imaging of the vasculature or the intravascular images. An electronic apparatus within the system (e.g., PC, dedicated hardware, or firmware) such as the host workstation 433 stores the three dimensional image in a tangible, non-transitory memory and renders an image of the 3D tissues on the display 380. In some embodiments, the 3D images will be coded for faster viewing. In certain embodiments, systems of the invention render a GUI with elements or controls to allow an operator to interact with three dimensional data set as a three dimensional view. For example, an operator may cause a video affect to be viewed in, for example, a tomographic view, creating a visual effect of travelling through a lumen of vessel (i.e., a dynamic progress view). In other embodiments an operator may select points from within one of the images or the three dimensional data set by choosing start and stop points while a dynamic progress view is displayed in display. In other embodiments, a user may cause an imaging catheter to be relocated to a new position in the body by interacting with the image.

In some embodiments, a user interacts with a visual interface and puts in parameters or makes a selection. Input from a user (e.g., parameters or a selection) are received by a processor in an electronic device such as, for example, host workstation 433, server 413, or computer 449. The selection can be rendered into a visible display. In some embodiments, an operator uses host workstation 433, computer 449, or terminal 467 to control system 400 or to receive images. An image may be displayed using an I/O 454, 437, or 471, which may include a monitor. Any I/O may include a keyboard, mouse or touch screen to communicate with any of processor 421, 459, 441, or 475, for example, to cause data to be stored in any tangible, nontransitory memory 463, 445, 479, or 429. Server 413 generally includes an interface module 425 to effectuate communication over network 409 or write data to data file 417. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections). In certain embodiments, host workstation 433 and imaging engine 855 are included in a bedside console unit to operate system 400.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell networks (3G, 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include programming language known in the art, including, without limitation, C, C++, Perl, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment) into patterns of magnetization by read/write heads, the patterns then representing new collocations of information desired by, and useful to, the user. In some embodiments, writing involves a physical transformation of material in tangible, non-transitory computer readable media with certain properties so that optical read/write devices can then read the new and useful collocation of information (e.g., burning a CD-ROM). In some embodiments, writing a file includes using flash memory such as NAND flash memory and storing information in an array of memory cells include floating-gate transistors. Methods of writing a file are well-known in the art and, for example, can be invoked automatically by a program or by a save command from software or a write command from a programming language.

In certain embodiments, display 380 is rendered within a computer operating system environment, such as Windows, Mac OS, or Linux or within a display or GUI of a specialized system. Display 380 can include any standard controls associated with a display (e.g., within a windowing environment) including minimize and close buttons, scroll bars, menus, and window resizing controls. Elements of display 380 can be provided by an operating system, windows environment, application programming interface (API), web browser, program, or combination thereof (for example, in some embodiments a computer includes an operating system in which an independent program such as a web browser runs and the independent program supplies one or more of an API to render elements of a GUI). Display 380 can further include any controls or information related to viewing images (e.g., zoom, color controls, brightness/contrast) or handling files comprising three-dimensional image data (e.g., open, save, close, select, cut, delete, etc.). Further, display 380 can include controls (e.g., buttons, sliders, tabs, switches) related to operating a three dimensional image capture system (e.g., go, stop, pause, power up, power down).

In certain embodiments, display 380 includes controls related to three dimensional imaging systems that are operable with different imaging modalities. For example, display 380 may include start, stop, zoom, save, etc., buttons, and be rendered by a computer program that interoperates with IVUS, OCT, or angiogram modalities. Thus display 380 can display an image derived from a three-dimensional data set with or without regard to the imaging mode of the system.

Alternatively, an imaging data set may be assessed, analyzed, and transformed with a system such as the system shown in FIG. 5, comprising CPU 1510, storage 1520, and monitor 1530. Storage 1520 may contain instructions for carrying out methods of the invention, e.g., to configure CPU 1510 to analyze the imaging data set for a parameter, assign an indicator to the medical device based on the presence of the parameter, and display the indicator on monitor 1530. For example CPU 1510 may direct monitor 1530 to display a longitudinal image of a lumen with a color-coded stent. In some embodiments, a system of the invention will additionally comprise graphical user interface (GUI) 1540, which allows a user to interact with the images. In some embodiments, CPU 1510, storage 1520, and monitor 1530 may be encompassed within system 400.

The systems and methods of use described herein can be performed using any type of computing device, such as a computer, that includes a processor or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Any target can be imaged by methods and systems of the invention including, for example, bodily tissue. In certain embodiments, systems and methods of the invention image within a lumen of tissue. Various lumen of biological structures may be imaged including, but not limited to, blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, vagina, uterus and fallopian tubes, structures of the urinary tract including urinary collecting ducts, renal tubules, ureter, and bladder, and structures of the head and neck and pulmonary system including sinuses, parotid, trachea, bronchi, and lungs.

Exemplary step-by-step methods that are used by the system to identify and display key information are described schematically in FIG. 6. It will be understood that each block of FIG. 6, as well as any portion of the systems and methods disclosed herein, can be implemented by computer program instructions. These program instructions may be provided to a processor to produce a machine, such that the instructions, which execute on the processor, create means for implementing the actions specified in the FIG. 6 or described for the systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes or even in a different sequence than illustrated without departing from the scope or spirit of the invention.

A basic function of a system of the invention is described in FIG. 6 in which an image data set is received, one or more parameters is specified and analyzed, an indicator is selected, and the indicator is displayed. In some instances, a threshold value of the parameter will be defined by the user, however in other instances this is not necessary. Additionally, the user may be provided with a GUI to set a threshold alert and interact with the images, thereby triggering an alert when the threshold value is exceeded. In alternative embodiments, a user may also cause parameter values to be displayed or cause additional images to be displayed by interacting with the GUI.

A system of the invention is capable of imaging a biological lumen, assessing properties of the lumen, and then displaying the collected information in an easy-to-read format. For instance, as shown in FIG. 7, a system of the invention is capable of evaluating the flow within a blood vessel. In FIG. 7, the false (red) color in the interior of the lumen outside the guidewire image (central circle) is indicative of healthy blood flow. In some embodiments, the images will be displayed in real time and may oscillate in color or shade to communicate information regarding flow, pressure, temperature, velocity, or direction, among other information. In some embodiments, the system will include a button on the keyboard or the GUI that allows a user to turn the information off and on.

In alternative embodiments, the system can be used to evaluate the placement of a device, such as a stent, as shown in FIG. 8. Using a system of the invention, an IVUS image of a portion of a vessel with a placed stent is collected with a guidewire, and the image processing components produce an image showing a cut-away of the vessel including arms of a stent. As shown in FIG. 8, the stent is malapposed, i.e., portions of the stent are not touching the luminal wall. Malapposed stents can further exacerbate cardiovascular issues because the pocket between the lumen wall and the stent fills with plaque or cells, greatly reducing blood flow through the region.

The guidewires, methods, and systems of the invention may be used in the treatment of a number of disorders in a subject. For example, the guidewires, methods, and systems can be used to treat a variety of vascular diseases, including, but not limited to, atherosclerosis, ischemia, coronary blockages, thrombi, occlusions, stenosis, and aneurysms. The guidewires, methods, and systems can be used to access and treat a large number of locations that are accessible via the vasculature or urological or reproductive tracts. Such locations include the heart, brain, lungs, liver, kidneys, prostate, ovaries, testes, gallbladder, pancreas, and lymph nodes, among other locations. The guidewires, methods, and systems can be used to treat a variety of diseases, including cardiovascular disease, cancer, inflammatory disease (e.g., autoimmune disease, arthritis), pain, and genetic disorders.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for measuring and displaying a characteristic of a biological lumen, comprising a sensing guidewire, a processor, memory, and a display, operatively connected together, wherein:
the sensing guidewire comprises
a first optical fiber comprising a first blazed Bragg grating, the grating being at least partially reflective of a first wavelength, and
a photoabsorptive member that absorbs the first wavelength and is in photocommunication with the first blazed Bragg grating; and
the memory comprises instructions that when executed cause the processor to receive data corresponding to a luminal measurement taken with the sensing guidewire and cause the display to display an image of the lumen with the luminal measurement represented in the image.

2. The system of claim 1, wherein the photoabsorptive member is in acoustic communication with an exterior of the sensing guidewire.

3. The system of claim 2, wherein photoabsorption of the first wavelength by the photoabsorptive member creates acoustic waves in proximity to the sensing guidewire.

4. The system of claim 1, wherein the sensing guidewire further comprises:
a second optical fiber comprising a second blazed Bragg grating being at least partially reflective of a second wavelength; and
a photoreflective member that reflects the second wavelength and is in photocommunication with the second blazed Bragg grating.

5. The system of claim 4, wherein acoustic waves in proximity to the sensing guidewire cause a deflection of the photoreflective member.

6. The system of claim 5, wherein deflection of the photoreflective member creates a change in a pathlength for the second wavelength between the second blazed Bragg grating and the photoreflective member.

7. The system of claim 4, wherein the first and second wavelengths are the same wavelength.

8. The system of claim 1, wherein the sensing guidewire further comprises a strength member.

9. The system of claim 8, wherein the strength member is a coil.

10. The system of claim 9, wherein the coil comprises Nitinol.

11. The system of claim 1, wherein the diameter of the sensing guidewire is 3 mm (9 French) or less.

12. The system of claim 1, wherein the luminal measurement comprises a cross-sectional dimension of the lumen.

13. The system of claim 1, wherein the luminal measurement is blood flow through the lumen.

14. The system of claim 1, wherein the luminal measurement is a location of an implanted structure within the lumen.

15. The system of claim 14, wherein the implanted structure is a stent.

16. The system of claim 1, wherein the luminal measurement is represented with a color.

17. The system of claim 1, wherein the sensing guidewire further comprises a pressure sensor.
